(19) United States
(12) Patent Application Publication
(10) Pub. No.: US 2015/0276700 A1
(43) Pub. Date: Oct. 1, 2015

(54) REPRESENTING THE TASTE OF FOOD

(71) Applicant: TOSHIBA GLOBAL COMMERCE SOLUTIONS HOLDINGS CORPORATION, Tokyo (JP)

(72) Inventors: Vibhor GOEL, Bangalore (IN); Kavita PRASAD, Bangalore (IN); Megha SAKRIKAR, Bangalore (IN)

(73) Assignee: TOSHIBA GLOBAL COMMERCE SOLUTIONS HOLDINGS CORPORATION, Tokyo (JP)

(21) Appl. No.: 14/227,647

(22) Filed: Mar. 27, 2014

(51) Int. Cl. G01N 33/02

(52) U.S. Cl. CPC G01N 33/02

(57) ABSTRACT

Systems, methods, and computer program products to perform an operation comprising receiving selection of a food, determining a measure of similarity between the selected food and a plurality of taste preferences, wherein the measure of similarity is based at least in part on a plurality of taste attributes of the selected food, wherein each attribute of the plurality of taste attributes specifies a respective magnitude of a fundamental taste, of a plurality of fundamental tastes, and wherein each taste preference in the plurality of taste preferences specifies a respective preference for each fundamental taste, and upon determining that the measure of similarity is within a similarity threshold, displaying an indication that the selected food is preferable relative to the plurality of taste preferences.

### BACKGROUND

The present disclosure relates to computer software, and more specifically, to computer software to represent the taste of food.

It is difficult to determine how a food or drink will taste, especially when a person has not tried a particular food or drink in the past. Moreover, product packaging (if any) does not provide consumers with specific information indicating how the food or drink may taste, beyond general descriptors such as “sweet” or “spicy.” As such, consumers may be unwilling to experiment with new foods and drinks, due to concern of purchasing a product with taste the consumers dislike.

### SUMMARY

Embodiments disclosed herein include at least a system, method, and computer program product to perform an operation comprising receiving selection of a food, determining a measure of similarity between the selected food and a plurality of taste preferences, wherein the measure of similarity is based at least in part on a plurality of taste attributes of the selected food, wherein each attribute of the plurality of taste attributes specifies a respective magnitude of a fundamental taste, of a plurality of fundamental tastes, and wherein each taste preference in the plurality of taste preferences specifies a respective preference for each fundamental taste, and upon determining that the measure of similarity is within a similarity threshold, displaying an indication that the selected food is preferable relative to the plurality of taste preferences.

### BRIEF DESCRIPTION OF THE SEVERAL VIEWS OF THE DRAWINGS

FIGS. 1A-1C depict example outputs for representing the taste of food, according to embodiments disclosed herein.

FIG. 2 depicts a system to represent the taste of food, according to one embodiment.

FIG. 3 is a flow chart illustrating a method to represent the taste of food, according to one embodiment.

FIG. 4 is a flow chart illustrating a method to determine a measure of similarity between a taste profile of a selected food and preferences in a user profile.

### DETAILED DESCRIPTION

Embodiments disclosed herein provide techniques to represent the taste of food as a combination of taste parameters, such as the fundamental (or basic) tastes of sweet, savory, salty, sour, and umami. The combination of such taste parameters may be referred to herein as a “taste profile,” and could include values representing the magnitude of each fundamental taste in a given food or drink. For example, a five-digit code may be used to represent the taste profile, with each digit representing a magnitude for each fundamental taste of the food. For instance, if the magnitude ranges from 0-5, salt may have an example taste profile code of 00050. When a user selects a food (or drink), embodiments disclosed herein may retrieve and output the taste profile for the selected food. Furthermore, embodiments disclosed herein may compare the fundamental tastes in the taste profile of the selected food to preferences in one or more user profiles in order to determine a measure of similarity between the selected food and the user preferences. If the measure of similarity is within a predetermined threshold, embodiments disclosed herein may display an indication that the user may enjoy the taste of the selected food. Although the term “food” may be used herein as a reference example, this should not be considered limiting of the disclosure, as the disclosure applies equally to food and drink.

Stated differently, embodiments disclosed herein break the complex taste of a food into a scale of the five fundamental tastes. By representing food in such a manner, the complex, overall taste of a given food may be visualized as a magnitude (or degree) of each fundamental taste it has. Once the taste of a food item is broken up into a representation of the five fundamental tastes (the taste profile), the taste of the food item may be stored for future retrieval and displayed to customers via any type of computing device. Furthermore, the stored taste profile of different foods may be compared to user taste preferences stored in a user profile, thereby allowing users to scan foods and receive an indication as to whether they would like or dislike the taste of the food. Advantageously, users may scan barcodes or quick response (QR) codes for the food, which may be associated with the taste profile data, in order to determine, while shopping in a physical store, whether they may enjoy the taste of the food (based on personal opinion or a programmatically generated indication). Similarly, the taste profile of the food may be displayed on a product page in an online environment, such as an e-commerce site, in order to allow users to determine whether (based on personal opinion or the programmatically generated indication) they may enjoy the taste of the food.

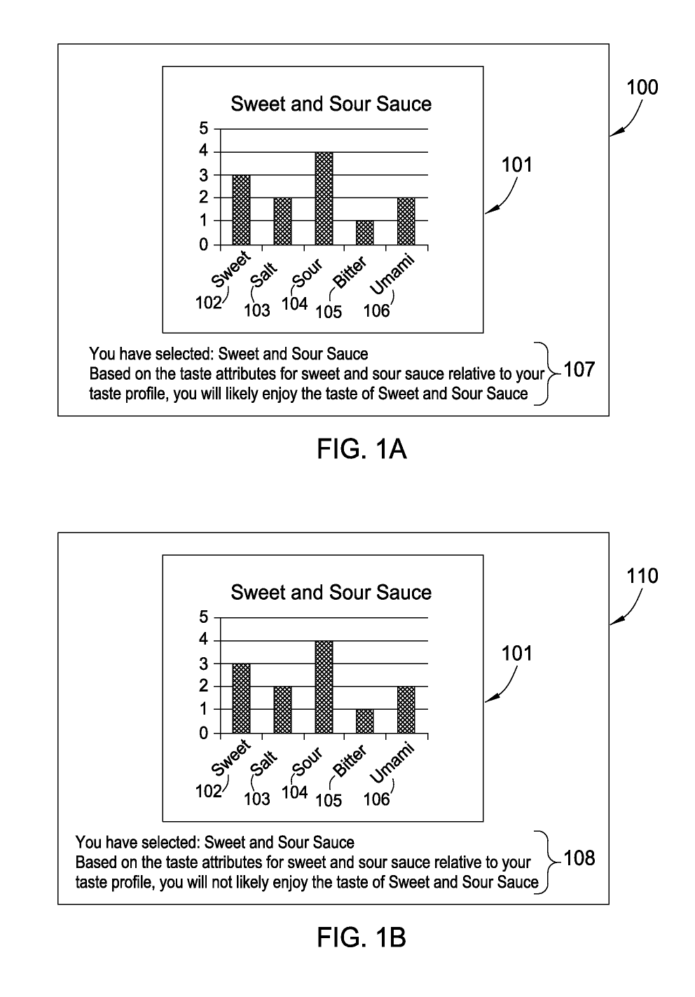

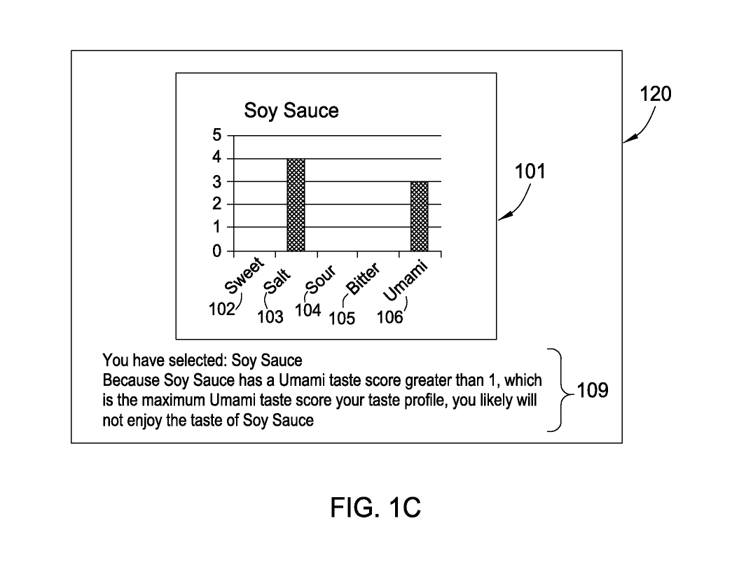

FIG. 1A depicts an example output 100 to represent the taste of food, according to one embodiment. Generally, the output 100 includes a taste profile 101 for a specific item, in this example, sweet and sour sauce. The output 100 may be generated and displayed, for example and without limitation, on the display of a mobile device in response to a user scanning a bar code or QR code of the bottle of sweet and sour sauce with the mobile device, such as a smart phone, tablet, and the like. As another example, the output 100 may be generated and displayed as part of a product page for the sweet and sour sauce on an online platform that can be accessed through a computing device. As shown, taste profile 101 indicates the magnitude of each of the fundamental tastes, namely sweet 102, salty 103, sour 104, bitter 105, and umami 106, for the sweet and sour sauce. In this embodiment, the taste profile 101 is displayed as a bar chart, however, the taste profile 101 may be represented in any feasible format, such as a table of values. As shown, the sweet and sour sauce has been defined to have, on a scale of zero to five, a sweet 102 magnitude of three, a salty 103 magnitude of two, a sour 104 magnitude of four, a bitter 105 magnitude of 1, and an umami 106 magnitude of two.

By outputting the taste profile 101 of the sweet and sour sauce, users may visualize the specific magnitude for each fundamental taste and make a decision as to whether they might like the sauce. Advantageously, a recommendation 107 also indicates the results of a comparison between the fundamental tastes in the taste profile 101 of the sweet and sour sauce and a set of user preferences in a user profile. The comparison may comprise computing a measure of similarity between the taste profile and the preferences in the user profile, and determining whether the measure of similarity is within a specified threshold. As shown, the comparison indicates that the user will likely enjoy the sweet and sour sauce. The comparison may be made by any feasible method or algorithm. For example, and without limitation, the user profile may indicate the user has a preference for both sweet and sour foods, both of which are present in the taste profile 101 of the sweet and sour sauce. As another example, the user profile may indicate that the user dislikes any foods with a bitterness 105 score higher than two. As still another example, the user profile may indicate that the user likes any food with an umami 106 score greater than 1. Continuing with the last example, based on the fact that the umami 106 score is greater than 1, embodiments disclosed herein may determine that the threshold value has been met, and that the user is likely to enjoy the taste of the sweet and sour sauce.

FIG. 1B depicts an example output 110 to represent the taste of food, according to one embodiment. The output 110 may be generated by scanning a bar code or QR code of the product with a mobile device, or by accessing an online page displaying the information for the product. Generally, any computing device may display the graphical outputs 100, 110, and 120, responsive to any request to display the outputs. As in FIG. 1A, the output 110 includes the taste profile 101 (again represented in a bar chart form) of the sweet and sour sauce, with unchanged values for the fundamental tastes 102-106. However, the recommendation 108 in FIG. 1B now indicates that the user is unlikely to enjoy the sweet and sour sauce. The recommendation 108 may be generated as a result of a comparison of the fundamental tastes in the taste profile 101 of the sweet and sour sauce and one or more user preferences in a user profile. The comparison may comprise computing a measure of similarity between the taste profile and the preferences in the user profile, and determining whether the measure of similarity is within a specified threshold.

As another example, and without limitation, the comparison may comprise computing a taste score for the sweet and sour sauce. The taste score may be computed by any feasible algorithm. For example, and without limitation, the taste score may be a weighted sum of each fundamental taste 102-106 in the taste profile 101. If the user does not like foods having a complex taste (for example, scores higher than two for at least three of the five fundamental tastes), the user profile may specify a taste threshold, that, when exceeded, indicates the taste is complex, and therefore is unlikely to be pleasant to the user. In another embodiment, a score may be computed for the preferences in the user profile, and compared to the taste score of the selected food. If the comparison indicates that the taste score is within a threshold value of the score of the preferences in the user profile, embodiments disclosed herein may output an indication that the user may enjoy the taste of the food. Conversely, if the score is not within the predefined threshold, embodiments disclosed herein may output an indication that the user may not enjoy the taste of the food.

As another example for computing the measure of similarity in FIG. 1B, the user may specify a dislike for sour foods in their user profile. In some embodiments, the profile may specify a threshold value, such as a sour 104 score greater than one. Since the sour 104 score in the taste profile 101 for the sweet and sour sauce is four, embodiments disclosed herein may determine that the maximum sour threshold has been exceeded, and that the user is unlikely to enjoy the sweet and sour sauce, because the measure of similarity between the user profile and the taste profile 101 is not within the specified threshold.

FIG. 1C depicts an example output 120 to represent the taste of food, according to one embodiment. The output 120 may be displayed, for example and without limitation, on the display of a mobile device in response to a user scanning a bar code or QR code of the bottle of sweet and sour sauce with the mobile device, such as a smart phone, tablet, and the like. As another example, the output 120 may be generated and displayed as part of a product page for the sweet and sour sauce on an online platform, accessed through a computing device. As shown, the output 120 includes a taste profile 101 for the soy sauce, indicating the soy sauce's magnitude of each of the fundamental tastes, namely sweet 102, salty 103, sour 104, bitter 105, and umami 106. As shown, the soy sauce has been defined to have, on a scale of zero to five, a sweet 102 magnitude of 0, a salty 103 magnitude of four, a sour 104 magnitude of zero, a bitter 105 magnitude of zero, and an umami 106 magnitude of three.

As shown, a recommendation 109 has been generated as a result of a comparison of the fundamental taste values 102-106 in the table 101 for the soy sauce and a set of user preferences in a user profile. The comparison may comprise computing a measure of similarity between the taste profile and the preferences in the user profile, and determining whether the measure of similarity is within a specified threshold. As shown, the recommendation 109 indicates that the user will likely not enjoy the taste of the soy sauce, as the soy sauce has an umami 106 score greater than 1, which the user has defined as the maximum umami threshold. As previously indicated, the results of the comparison may be based on any feasible method.

Generally, the taste profile 101 of each food item in FIGS. 1A-1C may be defined by any feasible method. For example, and without limitation, a user may define a taste profile for one or more foods or drink, and store the taste profiles at a storage location. As another example, and without limitation, the taste profile data may be programmatically determined by a system with sensors configured to detect each fundamental taste in the food or drink. Once the system is presented with a sample of the food or drink, the sensors can determine the magnitude of each fundamental taste in the food or drink, and store the taste profile at a storage location.

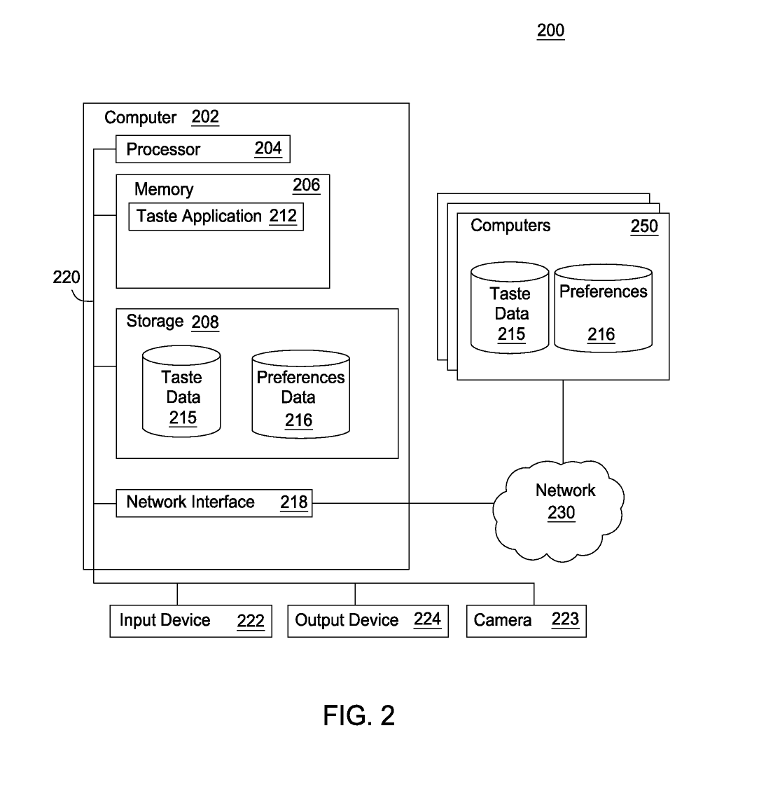

FIG. 2 depicts a system 200 to represent the taste of food, according to one embodiment. The networked system 200 includes a computer 202. The computer 202 may also be connected to other computers via a network 230. In general, the network 230 may be a telecommunications network and/or a wide area network (WAN). In a particular embodiment, the network 230 is the Internet.

The computer 202 generally includes a processor 204 connected via a bus 220 to a memory 206, a network interface device 218, a storage 208, an input device 222, and an output device 224. The computer 202 is generally under the control of an operating system (not shown). Examples of operating systems include the UNIX operating system, versions of the Microsoft Windows operating system, and distributions of the Linux operating system. (UNIX is a registered trademark of The Open Group in the United States and other countries. Microsoft and Windows are trademarks of Microsoft Corporation in the United States, other countries, or both. Linux is a registered trademark of Linus Torvalds in the United States, other countries, or both.) More generally, any operating system supporting the functions disclosed herein may be used. The processor 204 is included to be representative of a single CPU, multiple CPUs, a single CPU having multiple processing cores, and the like. The network interface device 218 may be any type of network communications device allowing the computer 202 to communicate with other computers via the network 230.

The storage 208 may be a persistent storage device. Although the storage 208 is shown as a single unit, the storage 208 may be a combination of fixed and/or removable storage devices, such as fixed disc drives, solid state drives, SAN storage, NAS storage, removable memory cards or optical storage. The memory 206 and the storage 208 may be part of one virtual address space spanning multiple primary and secondary storage devices.

The input device 222 may be any device for providing input to the computer 202. For example, a keyboard and/or a mouse may be used. The output device 224 may be any device for providing output to a user of the computer 202. For example, the output device 224 may be any conventional display screen or set of speakers. Although shown separately from the input device 222, the output device 224 and input device 222 may be combined. For example, a display screen with an integrated touch-screen may be used. The camera 223 is a camera configured to capture image data, which may be used by the processor 204 to identify an identifier of a scanned product, such as a bar code or a QR code on the label of a food or drink item.

As shown, the memory 206 contains the taste application 212, which is an application generally configured to define, for any given food or drink, a taste profile composed of the five fundamental tastes. The taste application 212 is further configured to associate the taste profile with the food or drink, such that the taste application 212 may subsequently display the taste profile of a food or drink selected by a user. The taste application 212 is further configured to compare the taste profile of a selected food or drink, which may be stored in the taste data 215, with user profile preferences of one or more users stored in the preferences 216. Generally, the taste application 212 may compare the taste profile with the user preferences in any feasible manner in order to return a result indicative of whether or not the user may like the taste of the food or drink. For example, the taste application 212 may compute a measure of similarity between the taste profile in the taste data 215 and the user preferences 216. If the taste application 212 determines that the user may enjoy the taste of the selected food based on the measure of similarity being within a predefined similarity threshold, the taste application 212 may also output promotional offers or coupons for the food (or another food that the taste application 212 determines that the user might enjoy). The similarity threshold may be pre-programmed in the taste application 212 (or stored in the storage 208), and may be of any type or format.

As shown, storage 208 contains the taste data 215, which stores taste profiles for a plurality of different foods. The taste profiles in the taste data 215 may include, without limitation, a magnitude of each fundamental taste in a food or drink, as well as a product identifier of the food or drink (such as a standard bar code or QR code). The taste profiles in the taste data 215 may be defined by user input, or programmatically defined by a system configured to detect, via one or more sensors, the magnitude of each fundamental taste in a food or drink. The taste profiles in the taste data 215 may be represented according to any predefined format and scale sufficient to show a range of values for each fundamental taste. For example, the magnitude of each fundamental taste may be stored as a value in the range of 0-1, 0-5, 0-10, and so on. For example, the taste profile of sugar may indicate a sweetness magnitude score of three out of five, while having scores of zero for the magnitude of salty, sour, savory, and umami.

The preference data 216 is generally configured to store user preference data for food and drink. The preference data 216 may include explicit taste likes and dislikes, as well as a number of rules related to their preferences. For example, a user may love the taste of umami, and may specify that any food with an umami score greater than four should be returned as preferable, regardless of any other taste scores. Generally, any types of rules and preferences may be defined in the preference data 216. The users may be identified by a unique identifier in the preference data 216, and may specify a user-specific similarity threshold. In some embodiments, the preference data 216 may further include similarity thresholds for more than one user, or for all users. The user preference data may indicate those fundamental tastes (as well as any combination thereof) that the user likes or dislikes. The user preference data for the food and drink may be represented in any feasible format. For example, one user may indicate that they “like sweet foods,” while another user may indicate that they “like foods having a sweetness score of less than three but greater than zero.”

In some embodiments, the preference data 216 may include foods previously compared against the user profile, and that are known to be liked or disliked by the user. Doing so may allow the taste application 212 to rely on these historic recommendations in order to generate future recommendations. Generally, the taste application 212 may learn user preferences. For example, if the user indicates that they like a specific food, the taste application 212 may adjust the user preferences in the preference data 216 based on the specified food's taste profile in the taste data 215. Therefore, if the user repeatedly indicates that they like the taste of sweet desserts, the sweet value in the user's preferences in the preference data 216 may increase over time. In addition, the taste application 212 may learn the user's preferences based on buying habits over time. For example, if the user buys salty snacks every week, but does not indicate an explicit preference for salty foods, the taste application 212 may store an indication in the preference data 216 reflecting the user's repeated purchase, and therefore preference for, salty foods.

Furthermore, the taste application 212 may provide an interface configured to receive user feedback regarding the generated recommendation, which the taste application 212 may store in the preference data 216 in order to use in generating future recommendations. A plurality of computers 250 may store remote instances of the taste data 215 and the preference data 216, which may be remotely accessed by the taste application 212.

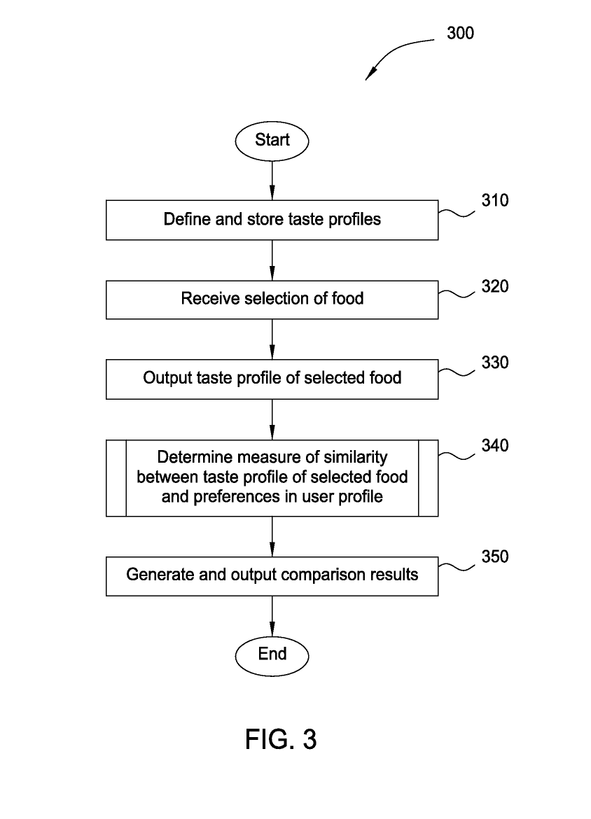

FIG. 3 is a flow chart illustrating a method 300 to represent the taste of food, according to one embodiment. Generally, the steps of the method 300 break down the taste of a food into a stored “taste profile” of fundamental tastes (sweet, sour, savory, salty, and umami), display the taste profile of a selected food to a user, and compare the taste profile of a selected food to a set of user preferences in order to generate a recommendation indicating whether or not the user will enjoy the taste of the food.

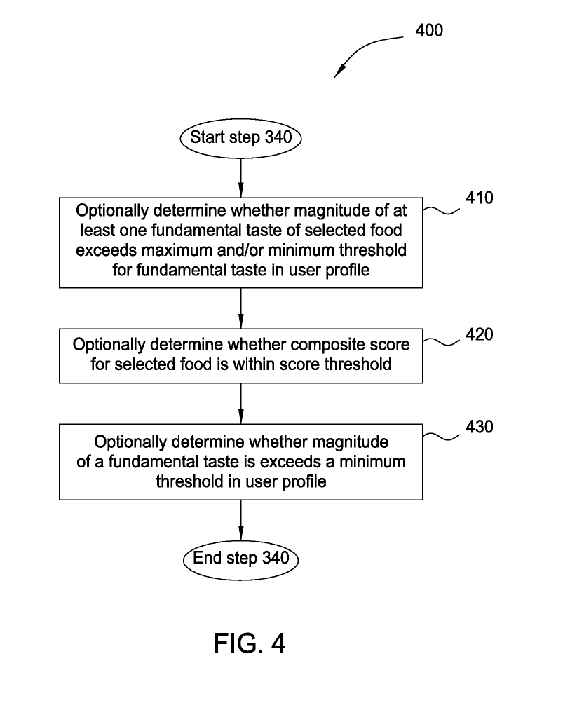

At step 310, a taste profiles for foods (and drinks) are defined. The taste profiles represent the taste of a food as a composite of each of the five fundamental tastes. The taste profiles may be created manually (e.g., a system administrator), or a system configured to sense the magnitude of each fundamental taste of a food or drink. The taste profiles may then be associated with a unique identifier of a food or drink (such as a bar code or QR code), and stored at a storage location for later retrieval. At step 320, the taste application 212 may receive user selection of a food. The taste application 212, for example, may be executing on a mobile device of the user, who scans the bar code or QR code of a food package. In another embodiment, the taste application 212 may be invoked as part of a web application which displays information about a food or drink that displayed on a web page. At step 330, the taste application 212 may output the taste profile of the selected food. The taste application 212 may output the taste profile in any feasible manner. For example, the taste profile may be represented as a bar chart, line chart, or a table including the magnitude of each fundamental taste for the selected food. At step 340, described in greater detail with reference to FIG. 4, the taste application 212 may determine a measure of similarity between the taste profile of the selected food to a set of preferences in a user profile, in order to generate a recommendation indicating whether or not the user may like the taste of the selected food. For example, if the measure of similarity is within a predetermined similarity threshold, the taste application 212 may generate an indication that the user may like the taste of the selected food. Conversely, the taste application 212 may generate an indication that the user is not likely to enjoy the taste of the food upon determining that the measure of similarity is not within the predefined similarity threshold. At step 350, the taste application 212 may generate and output results of the comparison, indicating whether or not the user may like the taste of the food.

FIG. 4 is a flow chart illustrating a method 400 corresponding to step 340 to determine a measure of similarity between a taste profile of a selected food and preferences in a user profile. Although depicted as a flow chart, one, several, or all of the steps of the method 400 may be invoked in any given iteration of the method 400. Furthermore, the disclosure is not limited to the specific comparison methods listed in FIG. 4, as any feasible scoring or comparison algorithms may be used to determine whether a user may like a food or drink in light of the taste profile data relative to the user preferences.

At step 410, the taste application 212 may optionally determine whether the magnitude of at least one fundamental taste for the selected food exceeds a maximum and/or minimum taste threshold for the fundamental taste in the user profile. In such an embodiment, the taste application 212 may ensure that the magnitudes of at least one (or each) fundamental taste in the taste profile of the food are within acceptable ranges specified in the user profile. For example, if the user specifies that they only like foods with scores greater than one (out of five, for example) for sweetness, saltiness, and umami, but scores of less than three for savory and sour, the taste application 212 may ensure that the at least one value in the taste profile falls above or below the specified thresholds. If at least one of the values in the taste profile is equal to or greater the corresponding value in the taste profile, the taste application 212 may determine that the measure of similarity is within the acceptable threshold, and determine that the user may like the taste of the food.

In other embodiments, the taste application 212 may ensure that a threshold count of fundamental tastes have values falling above or below the specified thresholds. For example, the threshold count may be two of the five fundamental tastes matching the preferences in the user profile. In such embodiments, for example, if three of five fundamental tastes are above or below the specified thresholds, the taste application 212 may determine that the measure of similarity between the taste profile and the user preferences is within the specified threshold. Therefore, if the food has scores of greater than one for sweet, salty, and umami, but scores of greater than three for savory and sour, the taste application 212 may generate a recommendation indicating that the user may like the taste of the food.

At step 420, the taste application 212 may optionally determine whether a composite score for the selected food is within a score threshold in order to determine the measure of similarity. In such embodiments, the taste application 212 may generate a score representing the overall taste of the food. For example, and without limitation, the taste application 212 may score the food using a weighted sum (or average) of each fundamental taste. Generally, any feasible method may be used to compute a score for the food, and likewise, any range, threshold, or other profile data may be stored in order to reflect user preferences.

At step 430, the taste application 212 may optionally determine whether a magnitude of a fundamental taste exceeds a dislike threshold for the fundamental taste specified in the user profile. In such embodiments, the taste application 212 ensures that a food, that may otherwise satisfy one or more user preferences, is not recommended due to the fact that a specified “dislike” of the user is satisfied. For example, a user may dislike all sour foods, which may be reflected in their user profile. Furthermore, the user may have one or more rules specified in their user profile indicating that any food having a sour score greater than zero in the taste profile is not preferable. Therefore, if the magnitude of the sour score in the taste profile is greater than zero, the taste application 212 will determine that the measure of similarity between the taste profile and the user preferences is not within the specified threshold, and indicate that the user is not likely to enjoy the taste of the food, regardless of the outcome of any other comparisons.

Advantageously, embodiments disclosed herein generate taste profiles representing the five fundamental tastes of food or drinks. Furthermore, embodiments disclosed herein may display these taste profiles responsive to user selection of the food through a computing interface. Further still, embodiments disclosed herein may compare the data in the taste profile of a selected food to one or more user profiles in order to generate and display a recommendation indicating whether the users may like or dislike the taste of the food or drink.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

As will be appreciated by one skilled in the art, aspects of the present disclosure may be embodied as a system, method or computer program product. Accordingly, aspects of the present disclosure may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present disclosure may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present disclosure may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present disclosure are described below with reference to flowchart illustrations and/or block diagrams of methods, apparatus (systems) and computer program products according to embodiments of the disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Embodiments of the disclosure may be provided to end users through a cloud computing infrastructure. Cloud computing generally refers to the provision of scalable computing resources as a service over a network. More formally, cloud computing may be defined as a computing capability that provides an abstraction between the computing resource and its underlying technical architecture (e.g., servers, storage, networks), enabling convenient, on-demand network access to a shared pool of configurable computing resources that can be rapidly provisioned and released with minimal management effort or service provider interaction. Thus, cloud computing allows a user to access virtual computing resources (e.g., storage, data, applications, and even complete virtualized computing systems) in “the cloud,” without regard for the underlying physical systems (or locations of those systems) used to provide the computing resources.

Typically, cloud computing resources are provided to a user on a pay-per-use basis, where users are charged only for the computing resources actually used (e.g. an amount of storage space consumed by a user or a number of virtualized systems instantiated by the user). A user can access any of the resources that reside in the cloud at any time, and from anywhere across the Internet. In context of the present disclosure, a user may access applications, such as the taste application 212, or related data available in the cloud. For example, the taste application 212 could execute on a computing system in the cloud and generate taste recommendations for selected foods or drinks. In such a case, the taste application 212 could generate the recommendations and store the recommendations, user profiles, and taste profiles at a storage location in the cloud. Doing so allows a user to access this information from any computing system attached to a network connected to the cloud (e.g., the Internet).

The flowchart and block diagrams in the Figures illustrate the architecture, functionality, and operation of possible implementations of systems, methods and computer program products according to various embodiments of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

While the foregoing is directed to embodiments of the present disclosure, other and further embodiments of the disclosure may be devised without departing from the basic scope thereof, and the scope thereof is determined by the claims that follow.

What is claimed is:

1. A method, comprising:
receiving a selection of a food;
determining a measure of similarity between the selected food and a plurality of taste preferences, wherein the measure of similarity is based at least in part on a plurality of taste attributes of the selected food, wherein each attribute of the plurality of taste attributes specifies a respective magnitude of a fundamental taste, of a plurality of fundamental tastes, and wherein each taste preference in the plurality of taste preferences specifies a respective preference for each fundamental taste; and
upon determining that the measure of similarity is within a similarity threshold, displaying, by operation of one or more computer processors, an indication that the selected food is preferable relative to the plurality of taste preferences.

2. The method of claim 1, further comprising:
upon determining that the measure of similarity is not within the similarity threshold, displaying an indication that the selected food is not preferable relative to the plurality of taste preferences.

3. The method of claim 1, wherein the plurality of taste preferences comprise user taste preferences in a user profile, wherein the plurality of fundamental tastes comprise: (i) sweet, (ii) salty, (iii) savory, (iv) sour, and (v) umami.

4. The method of claim 3, wherein determining the measure of similarity comprises:
comparing the magnitude of at least one fundamental taste in the set of taste attributes of the selected food to the respective taste preference for the corresponding fundamental taste; and
determining whether the magnitude of the at least one fundamental taste of the selected food is within a fundamental taste threshold for the respective taste preference.

5. The method of claim 3, wherein determining the measure of similarity comprises:
determining that a count of the magnitudes of each fundamental taste of the selected food that are within a fundamental taste threshold for respective taste preference exceeds a count threshold.

6. The method of claim 3, wherein determining the measure of similarity comprises:
determining whether a magnitude of a first fundamental taste in the plurality of attributes of the selected food exceeds a maximum threshold specified in the plurality of taste preferences for the first fundamental taste.

7. The method of claim 1, further comprising:
defining a set of taste attributes for each of a plurality of foods, including the food.

8. A system, comprising:
one or more computer processors; and
a memory containing a program, which when executed by the one or more computer processors, performs an operation comprising:
receiving selection of a food;
determining a measure of similarity between the selected food and a plurality of taste preferences, wherein the measure of similarity is based at least in part on a plurality of taste attributes of the selected food, wherein each attribute of the plurality of taste attributes specifies a respective magnitude of a fundamental taste, of a plurality of fundamental tastes, and wherein each taste preference in the plurality of taste preferences specifies a respective preference for each fundamental taste; and
upon determining that the measure of similarity is within a similarity threshold, displaying an indication that the selected food is preferable relative to the plurality of taste preferences.

9. The system of claim 8, the operation further comprising:
upon determining that the measure of similarity is not within the similarity threshold, displaying an indication that the selected food is not preferable relative to the plurality of taste preferences.

10. The system of claim 8, wherein the plurality of taste preferences comprise user taste preferences in a user profile, wherein the plurality of fundamental tastes comprise: (i) sweet, (ii) salty, (iii) savory, (iv) sour, and (v) umami.

11. The system of claim 10, wherein comparing the set of taste attributes to the set of taste preferences comprises:
comparing the magnitude of at least one fundamental taste in the set of taste attributes of the selected food to the respective taste preference for the corresponding fundamental taste; and
determining whether the magnitude of the at least one fundamental taste of the selected food is within a fundamental taste threshold for the respective taste preference.

12. The system of claim 10, wherein determining the measure of similarity comprises:
determining that a count of the magnitudes of each fundamental taste of the selected food that are within a fundamental taste threshold for respective taste preference exceeds a count threshold.

13. The system of claim 8, wherein determining the measure of similarity comprises:
determining whether a magnitude of a first fundamental taste in the plurality of attributes of the selected food exceeds a maximum threshold specified in the plurality of taste preferences for the first fundamental taste.

14. The system of claim 8, the operation further comprising:
defining a set of taste attributes for each of a plurality of foods, including the selected food.

15. A computer program product, comprising:
a computer-readable storage medium having computer-readable program code embodied therewith, the computer-readable program code comprising:
computer-readable program code configured to receive selection of a food;
computer-readable program code configured to determine a measure of similarity between the selected food and a plurality of taste preferences, wherein the measure of similarity is based at least in part on a plurality of taste attributes of the selected food, wherein each attribute of the plurality of taste attributes specifies a respective magnitude of a fundamental taste, of a plurality of fundamental tastes, and wherein each taste preference in the plurality of taste preferences specifies a respective preference for each fundamental taste; and
computer-readable program code configured to, upon determining that the measure of similarity is within a similarity threshold, display an indication that the selected food is preferable relative to the plurality of taste preferences.

16. The computer program product of claim 15, further comprising:
computer-readable program code configured to, upon determining that the measure of similarity is not within the similarity threshold, display an indication that the selected food is not preferable relative to the plurality of taste preferences.

17. The computer program product of claim 15, wherein the plurality of taste preferences comprise user taste preferences in a user profile, wherein the plurality of fundamental tastes comprise: (i) sweet, (ii) salty, (iii) savory, (iv) sour, and (v) umami.

18. The computer program product of claim 17, wherein comparing the set of taste attributes to the set of taste preferences comprises:
comparing the magnitude of at least one fundamental taste in the set of taste attributes of the selected food to the respective taste preference for the corresponding fundamental taste; and
determining whether the magnitude of the at least one fundamental taste of the selected food is within a fundamental taste threshold for the respective taste preference.

19. The computer program product of claim 17, wherein determining the measure of similarity comprises:
determining that a count of the magnitudes of each fundamental taste of the selected food that are within a fundamental taste threshold for respective taste preference exceeds a count threshold.

20. The computer program product of claim 16, wherein determining the measure of similarity comprises:
determining whether a magnitude of a first fundamental taste in the plurality of attributes of the selected food exceeds a maximum threshold specified in the plurality of taste preferences for the first fundamental taste.
